(19) United States
(12) Patent Application Publication
(10) Pub. No.: US 2020/0069461 A1
(43) Pub. Date: Mar. 5, 2020

(54) COOLING TOOL

(71) Applicant: SHARP KABUSHIKI KAISHA, Sakai City, Osaka (JP)

(72) Inventors: DAISUKE SHINOZAKI, Sakai City (JP); YUKA UTSUMI, Sakai City (JP); SATORU MOTONAMI, Sakai City (JP)

(21) Appl. No.: 16/463,235

(22) PCT Filed: Nov. 16, 2017
(86) PCT No.: PCT/JP2017/041211

§ 371 Date: May 22, 2019

(30) Foreign Application Priority Data
Nov. 22, 2016 (JP) 2016-227269

(51) Int. Cl. A61F 7/10; G01N 21/80

(52) U.S. Cl. CPC A61F 7/10; G01N 21/80

(57) ABSTRACT

Provided is a cooling tool that keeps a cooling object cold by utilizing the latent heat of a cold storage material and can visualize the physical condition of the cooling tool, i.e., can indicate whether the use condition of the cooling tool is appropriate. For example, when leakage of the cold storage material occurs, the cooling tool indicates the leakage of the cold storage material and shows whether the temperature is appropriate for keeping the cooling object cold. The cooling tool keeps the cooling object cold by utilizing the latent heat of a freezable material and includes: the freezable material that undergoes phase change at a specific temperature; a cold storage layer including a first container portion that contains the freezable material; and a condition indicator that shows the physical condition of the freezable material.

### TECHNICAL FIELD

The present invention relates to a cooling tool used to keep a cooling object cold by utilizing the latent heat of a cold storage material.

### BACKGROUND ART

Cooling therapy called icing, cryotherapy, etc. is a known art. In the cryotherapy, a feverish body part or the whole human body is cooled using various methods. For example, cold air is applied to the human body, or a cooling material is brought into contact with human skin.

PTL 1 discloses a cooling material that can comfortably fit a human head with an improved sense of feel and is expected to have sufficient cooling performance. This cooling material includes a plurality of horizontally connected connectable members having thickness of 15 to 35 mm and a freezable member having a thickness of 5 to 15 mm that are vertically stacked and housed in an outer bag.

### CITATION LIST

### Patent Literature

PTL 1: Japanese Unexamined Patent Application Publication No. 7-95998

### SUMMARY OF INVENTION

### Technical Problem

When a cooling tool deteriorates considerably during long-term use, leakage of a solution in the cooling tool may occur. In PTL 1, no consideration is given to an alert etc. when the cooling material leaks from the packaging bag.

When the solution in the cooling tool is colorless and transparent, the solution cannot be distinguished from dew condensation and sweat, and it takes time until the leakage of the solution is noticed. A cooling tool used for the human body is required to have a structure that allows early detection of leakage of the solution. One method for early detection of leakage of the solution is to use a colored cold storage material to visualize the leakage of the cold storage material. However, a problem with this method is that clothing is colored with the cold storage material.

In addition to the above problem, the leakage of the solution results in a reduction in the amount of the solution in the cooling tool and causes a reduction in phase change time. Therefore, the cooling function of the cooling tool is impaired.

In the cryotherapy, a cooling material is used to cool an affected part. In this case, when the temperature of the cooling material is excessively low for the human body, it is difficult to use the cooling material for a long time, and the cooling time may be insufficient. One idea to address this issue is to dispose a cloth etc. between the cooling material and the skin to maintain the skin temperature appropriately. The cooling material disclosed in PTL 1 is used for the human body. However, no consideration is given to its thermophysical properties and the temperature range during use. Therefore, when the cold storage material is brought into direct contact with the human body, an excessive amount of heat may be removed from the human body.

Humans have a nerve called TRPA1 that functions to allow recognition of “pain” when the skin temperature is reduced to 17° C. or lower. Therefore, when the human body is cooled, it is necessary to pay sufficient attention to the skin temperature in order to prevent the nerve from functioning. It is difficult to use a cooling material that causes the skin temperature to be reduced to 17° C. or lower for a long time, and this cooling material may cause frostbite in some cases. No cold storage material designed on the premise that it is used for the human body is disclosed in PTL 1, and the TRPA1 described above may function. If this is the case, the cold storage material is not suitable for long-term use.

The purpose of and means for emergency treatment of acute traumatic inflammation considerably differ from the purpose of and means for rehabilitation and palliative care. However, cooling tools appropriately usable according to their intended purpose have not been proposed.

The present invention has been made in view of the foregoing circumstances, and it is an object to provide a cooling tool that keeps a cooling object cold by utilizing the latent heat of a cold storage material and can visualize the physical condition of the cooling tool, i.e., can indicate whether the use condition of the cooling tool is appropriate. For example, when leakage of the cold storage material occurs, the cooling tool indicates the leakage of the cold storage material and shows whether the temperature is appropriate for keeping the cooling object cold.

To achieve the above object, the present invention takes the following measures. A cooling tool according to one aspect of the present invention keeps a cooling object cold by utilizing the latent heat of a freezable material and includes: the freezable material that undergoes phase change at a specific temperature; a cold storage layer having a first container portion that contains the freezable material; and a condition indicator that shows the physical condition of the freezable material.

### Advantageous Effects of Invention

In the above aspect of the present invention, the condition indicator that shows the physical condition of the freezable material is provided. Therefore, the user can judge at a glance the condition of the freezable material, e.g., whether the freezable material is leaking from the cold storage layer or whether the temperature of the freezable material is appropriate for keeping the cooling object cold, so that the cooling tool in an appropriate condition can be used.

### BRIEF DESCRIPTION OF DRAWINGS

FIG. 1A is a perspective view schematically showing a cooling tool according to a first embodiment.

FIG. 1B is a side view schematically showing the cooling tool according to the first embodiment.

FIG. 2 is a diagram showing changes in the state of a cold storage material due to temperature change.

FIG. 3 is a table showing examples of a freezable material included in a cold storage layer.

FIG. 4 is a table showing examples of a non-freezable material included in a buffer layer.

FIG. 5A is an illustration showing the function of the cooling according to the first embodiment.

FIG. 5B is an illustration showing the function of another cooling tool according to the first embodiment.

FIG. 5C is an illustration showing the function of another cooling tool according to the first embodiment.

FIG. 6 is a table showing examples of pH indicators used in the first embodiment and the color change pH values of the pH indicators.

FIG. 7 is a table showing the difference in pH due to the difference in composition between freezable materials

FIG. 8 is a perspective view schematically showing a cooling tool 2 according to a second embodiment.

FIG. 9 is an illustration showing the concept of temperature ranges in the second embodiment.

FIG. 10A is an illustration showing an example of a label that uses a temperature display method using a thermochromic material (decolorizable type).

FIG. 10B is an illustration showing an example of a label that uses a temperature display method using a thermochromic material (color developing type).

FIG. 11 shows changes in temperature of a cold storage layer and changes in display using a thermochromic material.

FIG. 12 is an illustration showing an example of a combination of thermochromic materials.

FIG. 13 shows a method for producing the cooling tool.

FIG. 14A is an illustration showing a method for producing the cooling tool.

FIG. 14B is an illustration showing the method for producing the cooling tool.

FIG. 15A is an illustration showing an example of a cooling tool with warning members disposed on the cold storage layer and the buffer layer.

FIG. 15B is an illustration showing another example of the cooling tool with the warning members disposed on the cold storage layer and the buffer layer.

FIG. 15C is an illustration showing another example of the cooling tool with the warning members disposed on the cold storage layer and the buffer layer.

FIG. 16A is a perspective view schematically showing a cooling tool vacuum-packed with film.

FIG. 16B is a side view schematically showing the cooling tool vacuum-packed with the film.

FIG. 16C is a photograph showing an example of a small desktop vacuum packaging machine.

FIG. 16D is an illustration showing an example of the procedure of a protection method.

FIG. 17 is an illustration schematically showing a cooling tool according to a third embodiment.

FIG. 18 is an illustration showing leakage of the freezable material from the cold storage layer into the buffer layer.

FIG. 19A is an illustration showing a process of manufacturing the cooling tool according to the third embodiment.

FIG. 19B is an illustration showing the process of manufacturing the cooling tool according to the third embodiment.

FIG. 19C is an illustration showing another process of manufacturing the cooling tool according to the third embodiment.

FIG. 19D is an illustration showing the process of manufacturing the cooling tool according to the third embodiment.

FIG. 20A is an illustration schematically showing a cooling tool in a fourth embodiment.

FIG. 20B is an illustration showing an example of the use of the cooling tool in the fourth embodiment.

FIG. 21A is an illustration schematically showing the cooling tool that uses a fixing jig.

FIG. 21B is an illustration showing how the cooling tool produced using a supporter is attached to an arm.

### DESCRIPTION OF EMBODIMENTS

The present inventors have paid attention to the following issues. When leakage of a cold storage material contained in a cooling tool occurs, the cold storage material may adhere to a cooling object if the leakage of the cold storage material is unnoticed for a while, and the cooling function may be impaired. When the temperature of the cooling tool used for the cooling object is excessively low, a portion of the cooling object that is in contact with the cooling tool may be damaged. When the temperature of the cooling tool used for the cooling object is excessively high, the cooling effect is impaired. The inventors have found that, when a condition indicator is provided in the cooling tool to visualize the physical condition of the cooling tool, the physical condition of the cooling tool can be judged at a glance. Specifically, for example, whether the freezable material is leaking from the cold storage layer or whether the temperature of the freezable material is appropriate for keeping the cooling object cold can be judged at a glance, and therefore the cooling tool in an appropriate condition can be used. Thus, the present invention has been completed. Embodiments of the present invention will be specifically described with reference to the drawings.

### First Embodiment

### (Structure of Cooling Tool)

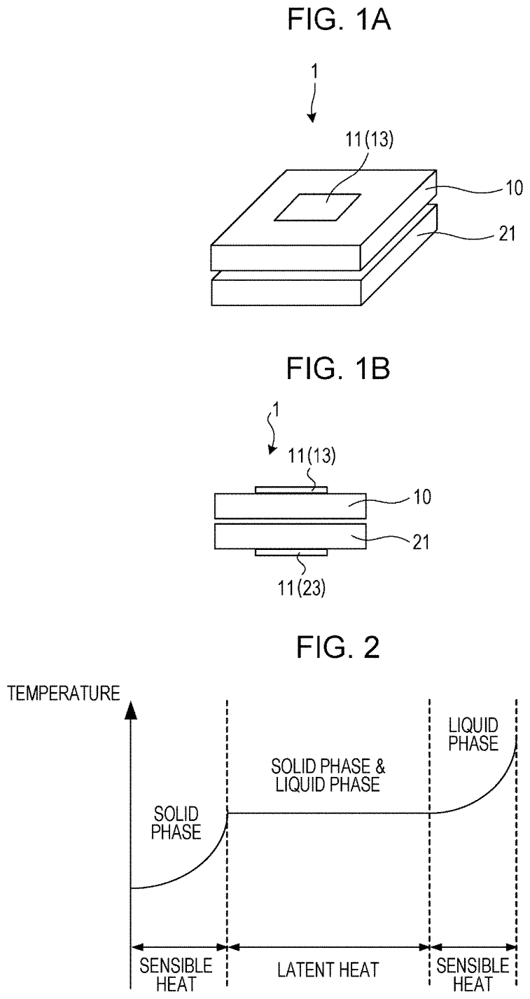

FIG. 1A is a perspective view schematically showing a cooling tool according to a first embodiment, and FIG. 1B is a side view schematically showing the cooling tool according to the first embodiment. The cooling tool 1 includes: a cold storage layer 10 including a freezable material that undergoes phase change at a specific temperature and a first container portion that contains the freezable material; a buffer layer 21 including a non-freezable material that forms a liquid phase at the phase change temperature of the freezable material and a second container portion that contains the non-freezable material; and condition indicators 11 (a first warning member 13 and a second warning member 23) each disposed at a given position on the cold storage layer or the buffer layer. In the following description of the cooling tool 1 according to the first embodiment, one of the condition indicators 11 disposed at a given position on the cold storage layer 10 is the first warning member 13, and the other one of the condition indicators 11 disposed at a given position on the buffer layer 21 is the second warning member 23.

The first warning member 13 is impregnated with a first indicator whose color changes according to the pH of the freezable material. When the freezable material comes into contact with the first warning member 13, the first warning member 13 changes its color. The second warning member 23 is impregnated with a second indicator whose color changes according to the pH of the non-freezable material. When the non-freezable material comes into contact with the second warning member 23, the second warning member 23 changes its color.

### (Cold Storage Layer and Buffer Layer)

FIG. 2 is a diagram showing changes in the state of the cold storage material due to temperature change. As shown in FIG. 2, when the temperature of the cold storage material is changed from low temperature to high temperature or from high temperature to low temperature, the cold storage material changes its state from a solid phase to a liquid phase or from a liquid phase to a solid phase. When only the solid phase or the liquid phase is present, sensible heat is generated based on the specific heat of the material.

The cold storage material undergoes phase change from a solid phase to a liquid phase or from a liquid phase to a solid phase through a two-phase coexistence state in which the solid and liquid phases coexist. When the temperature of the cold storage material reaches its melting point in the two-phase coexistence state, the cold storage material undergoes phase change from a solid phase to a liquid phase while absorbing heat and thereby melts. Alternatively, when the temperature of the cold storage material reaches its freezing point, the cold storage material undergoes phase change from a liquid phase to a solid phase while releasing heat and thereby solidifies. In the two-phase coexistence state in which the solid and liquid phases coexist, latent heat is generated. The freezable material and the non-freezable material used in the first embodiment are cold storage materials having the above-described properties.

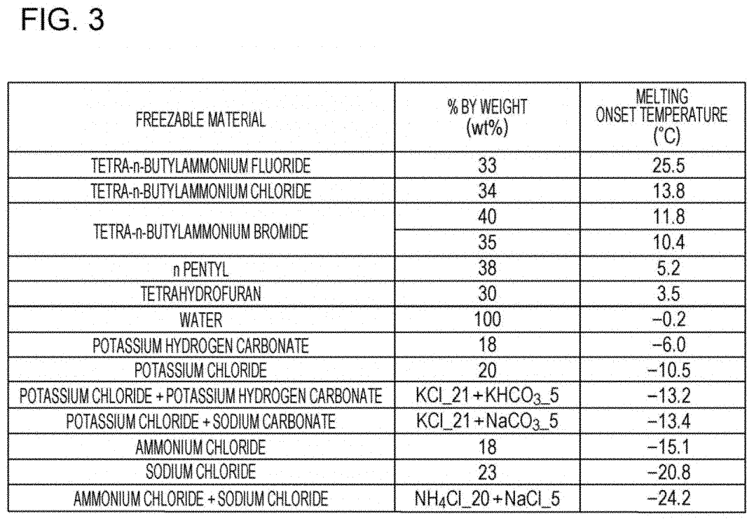

The cold storage layer 10 has thermal properties. FIG. 3 is a table showing examples of the freezable material contained in the cold storage layer. As shown in FIG. 3, the cold storage material used as the freezable material of the cold storage layer may be, for example, tetra-n-butylammonium fluoride with a concentration of 33 wt % or tetra-n-butylammonium chloride with a concentration of 34 wt %. The melting onset temperature of tetra-n-butylammonium bromide can be changed by adjusting its concentration, and such a material can be used as the cold storage material. A cold storage material suitable for the intended use may be used. The materials shown in FIG. 3 and used for the freezable material of the cold storage layer are merely examples, and the freezable material is not limited thereto.

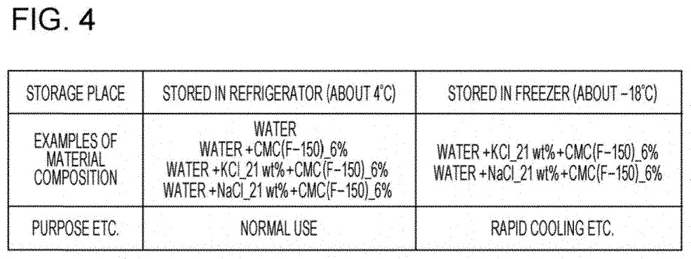

The buffer layer 21 has thermal properties and mechanical properties. First, the thermal properties of the buffer layer 21 will be described. FIG. 4 is a table showing examples of the non-freezable material contained in the buffer layer. As shown in FIG. 4, when, for example, the freezable material in the cold storage layer is frozen in a refrigerator (in the temperature range of about 3 to about 5° C.), “water,” “water+thickener (CMS(F-150)_6%),” “water+potassium chloride (KCl_21 wt %)+thickener (CMC(F-150)_6%),” or “water+sodium chloride (NaCl_23 wt %)+thickener (CMC(F-150)_6%)” is used as the non-freezable material in the buffer layer. Since the temperature range of the refrigerator is not lower than 0° C., the non-freezable material in the buffer layer does not freeze even if it is composed only of water.

However, for example, when the freezable material in the cold storage layer is subjected to rapid freezing in a freezer (temperature range: −18° C. to −20° C.), if the non-freezable material in the buffer layer is composed only of water, the buffer layer freezes because the temperature range of the freezer is lower than 0° C. When the freezer is used for freezing, “water+sodium chloride (NaCl_23 wt %)+thickener (CMC(F-150)_6%)” is used. In this case, by using the freezer (temperature range: −18° C. to −20° C.), the freezable material in the cold storage layer can be frozen faster than that in the refrigerator while the non-freezable material in the buffer layer does not freeze. Preferably, sodium chloride is used for the non-freezable material. The materials for the non-freezable material in the buffer layer shown in FIG. 4 are merely example, and the non-freezable material is not limited thereto. Any material that does not freeze in the freezer may be used.

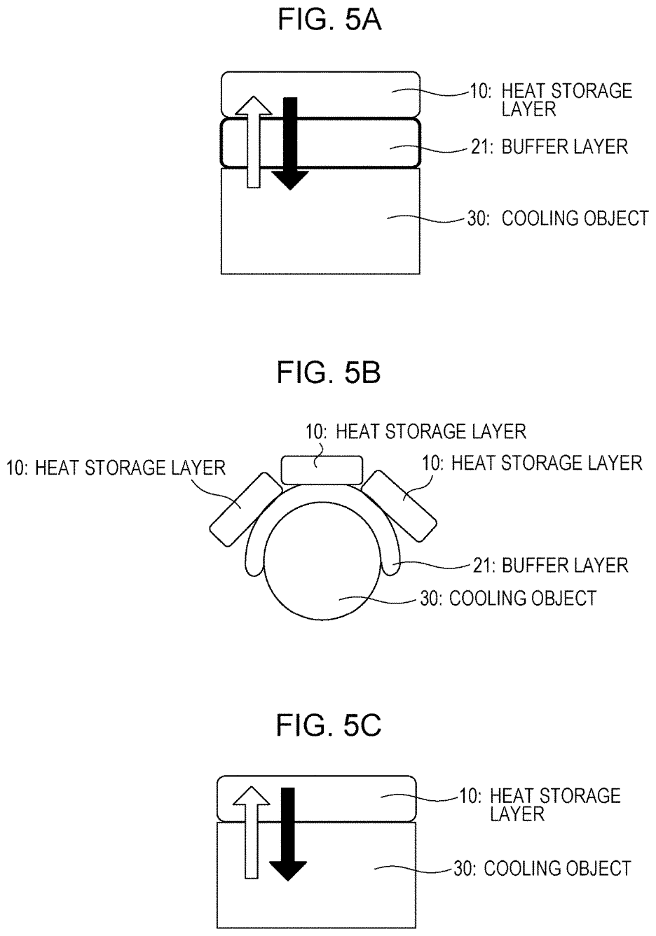

Next, the mechanical properties of the buffer layer 21 will be described. FIGS. 5A to 5C show the functions of cooling tools according to the first embodiment. As shown in FIG. 5A, the buffer layer is in contact with the cooling object and allows heat transfer between the cooling object and the cold storage layer. With this structure, heat transfer is mitigated (the amount of heat removed from the cooling object is reduced). The non-freezable material contained in the buffer layer forms a liquid phase at the phase change temperature of the freezable material, and a packaging material for the buffer layer is also flexible. Therefore, the adhesion of the buffer layer to the cooling object high, as shown in FIG. 5B. A plurality of cold storage layers and a plurality of buffer layers may be connected through a joint mechanism. To improve the adhesion between the cooling object and the buffer layer, the viscosity of the non-freezable material may be increased to allow the buffer layer to maintain its shape easily. By increasing the viscosity of the non-freezable material, spreading of the non-freezable material when leakage of the non-freezable material occurs can be reduced. As shown in FIGS. 5A and 5B, the buffer layer has not only the thermal function but also the mechanical function.

When it is unnecessary to mitigate heat transfer, e.g., when the cooling object is subjected to rapid cooling, the buffer layer may not be provided as shown in FIG. 5C, and the cold storage layer may be brought into direct contact with the cooling object.

### (Warning Members)

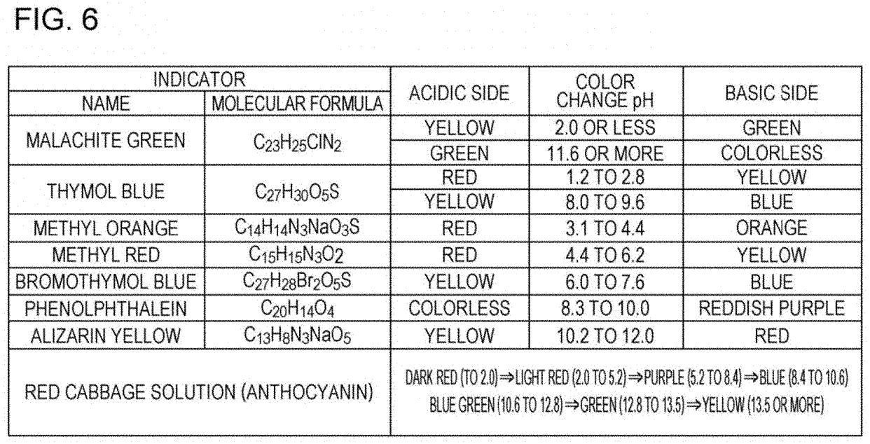

The first warning member and the second warning member (hereinafter referred to simply as warning members) are each composed of a pH indicator and a member (paper or cloth) impregnated with the pH indicator. FIG. 6 is a table showing examples of the pH indicators used for the cooling tool according to the first embodiment and the color change pH values of the pH indicators. In each warning member, its pH indicator changes color depending on acidity, neutrality, and alkalinity. When a warning member composed of a pH indicator and a member impregnated with the pH indicator is placed on the cold storage layer or the buffer layer, if the freezable material or the non-freezable material leaking from the cold storage layer or the buffer layer comes into contact with the warning member, the warning member changes its color.

The pH indicator in each warning member changes color according to the pH of the member itself (paper or cloth) impregnated with the indicator. Therefore, the indicator is selected in consideration of the relation between the indicator and the pH of a solution used in the cold storage layer or the buffer layer. When, for example, the freezable material is alkaline, a warning member (first warning member) impregnated with phenolphthalein used as the pH indicator is prepared. In this case, when the member itself (paper or cloth) impregnated with the pH indicator is acidic, the warning member (first warning member) is generally colorless. When a freezable material containing an alkaline solution comes into contact with the warning member (first warning member), the warning member (first warning member) turns reddish purple.

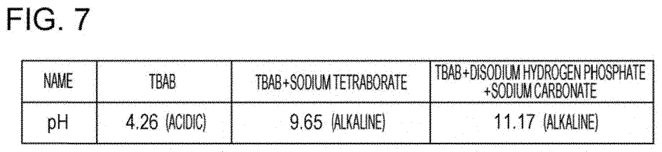

The pH value may be changed by a material added for the purpose of preventing instability of the freezing behavior of the freezable material caused by supercooling. A description will be given of the case in which, for example, a solution of tetrabutylammonium bromide (TBAB) shown in FIG. 7 is used as the freezable material.

An aqueous solution prepared by dissolving TBAB in water at 32 wt % (a 32 wt % aqueous TBAB solution) does not freeze in the temperature range of 0° C. or higher. To freeze this solution in a refrigerator (3 to 5° C.), it is necessary to add a supercooling inhibitor. To inhibit supercooling, “sodium tetraborate” or “disodium hydrogen phosphate+sodium carbonate” is mixed into the 32 wt % aqueous TBAB solution. This can inhibit supercooling, and stable freezing behavior can thereby be obtained.

As shown in FIG. 7, the 32 wt % aqueous TBAB solution containing only TBAB dissolved therein has a pH of 4.26 (acidic). However, an aqueous solution prepared by mixing “2% of sodium tetraborate” into the 32 wt % aqueous TBAB solution has a pH of 9.65, and an aqueous solution prepared by mixing “3% of disodium hydrogen phosphate+2% of sodium carbonate” into the 32 wt % aqueous TBAB solution has a pH of 11.17. These solutions are alkaline. These are added for the purpose of inhibiting supercooling. As described above, the pH value may be changed by a material added to the solution for some purpose. Such a change in the pH value may be utilized to cause a change in color. For example, a material that changes a neutral solution to acidic or alkaline may be added.

The freezable material and the non-freezable material may be aqueous solutions having different pH values. Therefore, a warning member impregnated with an indicator whose color changes according to the pH of the freezable material may be disposed on the cold storage layer, and a warning member impregnated with an indicator whose color changes according to the pH of the non-freezable material may be disposed on the buffer layer. When the freezable material in the cold storage layer and the non-freezable material in the freezable material are aqueous solutions having close pH values, warning members impregnated with the same indicator may be disposed.

In the description of the present embodiment, the tetrabutylammonium bromide (TBAB) solution is used as an example of the freezable material. The freezable material may be a quaternary ammonium salt solution containing tetrabutylammonium bromide (TBAB) but is not limited thereto. The same applies throughout the present description.

### Second Embodiment

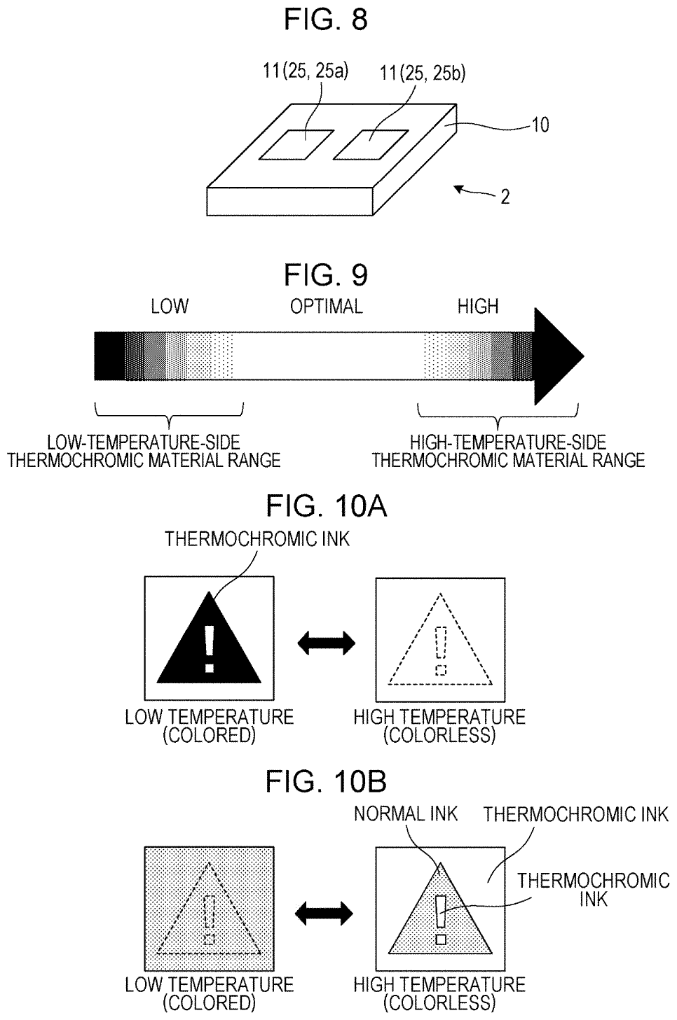

FIG. 8 is a perspective view schematically showing a cooling tool 2 according to a second embodiment. The cooling tool 2 includes a cold storage layer 10 and condition indicators 11 (temperature display members 25) disposed at given positions on the cold storage layer 10. The structure of the cold storage layer is the same as that in the first embodiment, and the description thereof will be omitted.

### (Temperature Display Members)

In the second embodiment, the temperature display members 25 formed from thermochromic materials will be described, but this is not a limitation. Any member that can measure and display temperature can be used. Each thermochromic material is a label-shaped thermochromic material that is applied to the surface of a packaging material for the cold storage layer, but this is not a limitation. For example, the thermochromic material may be contained in the cold storage material or may be applied to the packaging material for the cold storage layer by, for example, printing. The thermochromic material may be kneaded into the packaging material for the cold storage layer.

The thermochromic materials will be described. Each thermochromic material is a material whose color changes with temperature. There are various thermochromic materials with different temperature ranges, colors, forms, etc. Commercial forms of the thermochromic material are shown in Table 1.

TABLE 1

| | |
|---|---|
| Capsule | Water dispersion. Water colors and water-based inks can |
| slurry | be produced by mixing the capsule slurry with a binder. |
| | By impregnating plain T shirts with them, clothes whose |
| | color changes according to body temperature can be |
| | produced. |
| Capsule | Powder of fine particles of several micrometers. By mixing |
| powder | the powder with an oil-based binder, inks that can be |
| | printed on film, glass, metal, etc. can be manufactured. |
| Masterbatch | Product in the form of pellets of a plastic such as |
| | polypropylene. By subjecting the masterbatch with the |
| | amount of the resin increased 5 fold to 10 fold to injection |
| | molding, “bath toys,” “mugs with color changeable |
| | patterns,” “containers for managing the temperature of |
| | frozen foods,” “containers capable of displaying a color |
| | indicative of a suitable drinking temperature of cold drink,” |
| | etc. can be manufactured. The temperature of injection |
| | molding is 200° C. or lower. |
| Water-based | Suitable for printing on clothes such as T-shirts and most |
| screen ink | suitable for use with an 80 to 100 mesh screen. After |
| | printing, the ink is dried under heating at 110° C. to |
| | 120° C. for about 3 minutes and most suitably at 150° C. |
| | to 160° C. for about 1 minute. |
| Oil-based | Suitable for printing on plastic film etc. and most suitable |
| screen ink | for use with an 80 to 100 mesh screen. After printing, |
| | the ink is dried under heating most suitably at 40° C. |
| | to 60° C. for about 1 hour. |
| Water-based | Suitable for printing on paper, cloth, etc. After printing, the |
| ink | ink is dried under heating at 110° C. to 120° C. for about 3 |
| | minutes and most suitably at 150° C. to 160° C. for about 1 |
| | minute. |
| Oil-based | Suitable for printing on plastic film etc. After printing, the |
| ink | ink is dried under heating most suitably at 40° C. to 60° C. |
| | for about 3 minutes. |

FIG. 9 is an illustration showing the concept of temperature ranges in the second embodiment. In the concept of the temperature ranges shown in FIG. 9, a temperature range in which the cooling object is held at its suitable temperature is referred to as an optimal temperature range. A temperature range lower than the optimal temperature range is referred to as a low-temperature-side thermochromic material range, and a temperature range higher than the optimal temperature range is referred to as a high-temperature-side thermochromic material range. As shown in FIG. 8, a low-temperature-side temperature display member 25a and a high-temperature-side temperature display member 25b are disposed on the cold storage layer 10, and the temperature of the cold storage layer 10 can thereby be displayed.

FIGS. 10A and 10B are illustrations showing examples of a temperature display method using thermochromic materials. For example, FIG. 10A is an illustration showing an example of a decolorizable type label. At low temperature, a thermochromic material (thermochromic ink) in this label exhibits a color indicative of low temperature. At the suitable temperature, the thermochromic material turns colorless, and this indicates that the temperature is suitable. In “HIGH TEMPERATURE (COLORLESS)” in FIG. 10A, the contents of the label are shown by dotted lines. However, in practice, the contents of the label are not easily recognizable.

FIG. 10B is an illustration showing an example of a color change-type label. The label is coated with a thermochromic material (thermochromic ink) and also with a normal ink of the same color as the color of the thermochromic material at the suitable temperature. At the suitable temperature, portions coated with the thermochromic material exhibit the same color as the background ink and are blended into the background, and this indicates that the temperature is suitable. In “LOW TEMPERATURE (COLORED)” in FIG. 10B, the contents of the label are shown by dotted lines. However, in practice, the contents of the label are not easily recognizable. At high temperature, the portions coated with the thermochromic material exhibit a color different from the color of the normal ink (the thermochromic material is discolored), and this indicates that the temperature is high. In the above example of the temperature display method using the thermochromic materials, each thermochromic material exhibits a color at low temperature and turns colorless at high temperature. However, a thermochromic material that turns colorless at low temperature and exhibit a color at high temperature may be used.

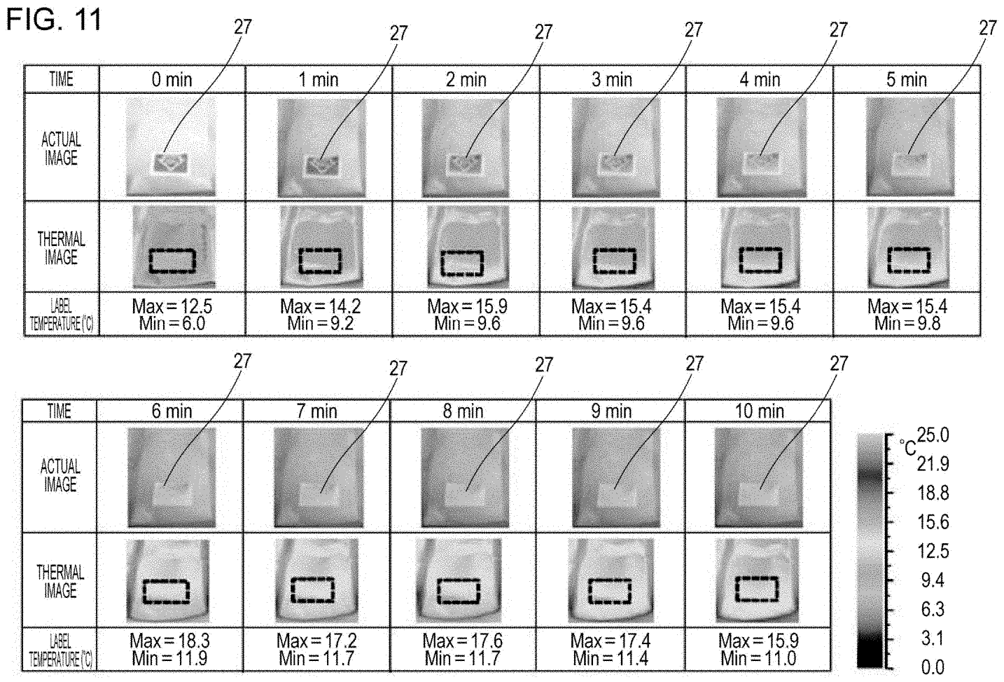

FIG. 11 shows changes in temperature of an actually packed TBAB cold storage layer with a label-shaped thermochromic material (decolorizable type) applied thereto and changes in display using the thermochromic material. As shown in FIG. 11, when the temperature of the cold storage layer is low (0 min), the thermochromic material thermochromic material in the label 27 exhibits a color indicative of low temperature. As time increases as 1, 2, . . . , 10 min, the temperature of the cold storage layer increases, and the thermochromic material in the label 27 gradually turns colorless. A mark on the label is merely an example and is not a limitation. Also, figures on the label are merely an example and do not mean anything.

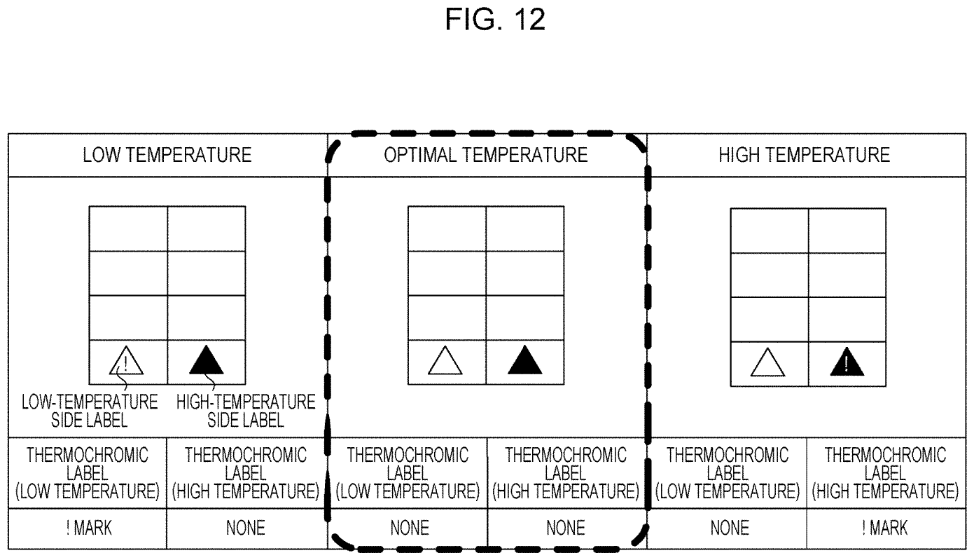

FIG. 12 shows an example of a combination of a decolorizable type thermochromic material and a color change-type thermochromic material. The decolorizable type thermochromic material is used in the low-temperature-side thermochromic material range, and the color change-type thermochromic material is used in the high-temperature-side thermochromic material range. As shown in FIG. 12, in the low-temperature-side thermochromic material range, a low-temperature-side thermochromic material label exhibits a color, and an “! mark,” for example, is displayed. In the high-temperature-side thermochromic material range, a high-temperature-side thermochromic material label exhibits a color, and an “! mark,” for example, is displayed. At the suitable temperature, the low-temperature-side thermochromic material label and the high-temperature-side thermochromic material label show no mark, and this indicates that the temperature is suitable. Any mark may be displayed on the labels, but a warning mark such as “!” or “×” is more preferable. When complementary colors are used for a combination of the background color of a label and the color exhibited by a thermochromic material (used for a warning mark portion), a high contrast is obtained, and the display can be enhanced.

In addition to the thermochromic materials, cholesteric liquid crystal seals may be used for the temperature display members 25. In the cholesteric liquid crystal, reflection color and transmission color vary depending on the period of its helical structure. Specifically, when a change in temperature or concentration occurs, the period is changed. Therefore, it is useful to use the cholesteric liquid crystal seals for the temperature display members.

In the second embodiment, as in the structure of the warning members in the first embodiment, a buffer layer may be provided, and a temperature display member used as a condition indicator may be disposed on the buffer layer. The cooling tool may include the warning members described in the first embodiment and also the temperature display members described in the second embodiment, although not illustrated.

### (Method for Producing Cooling Tool)

Next a method for producing the cooling tool 1, 2 configured as described above will be described.

(1) First, a method for producing the cold storage layer and the buffer layer included in the cooling tool will be described.

(a) Production Method Using Hand Sealing

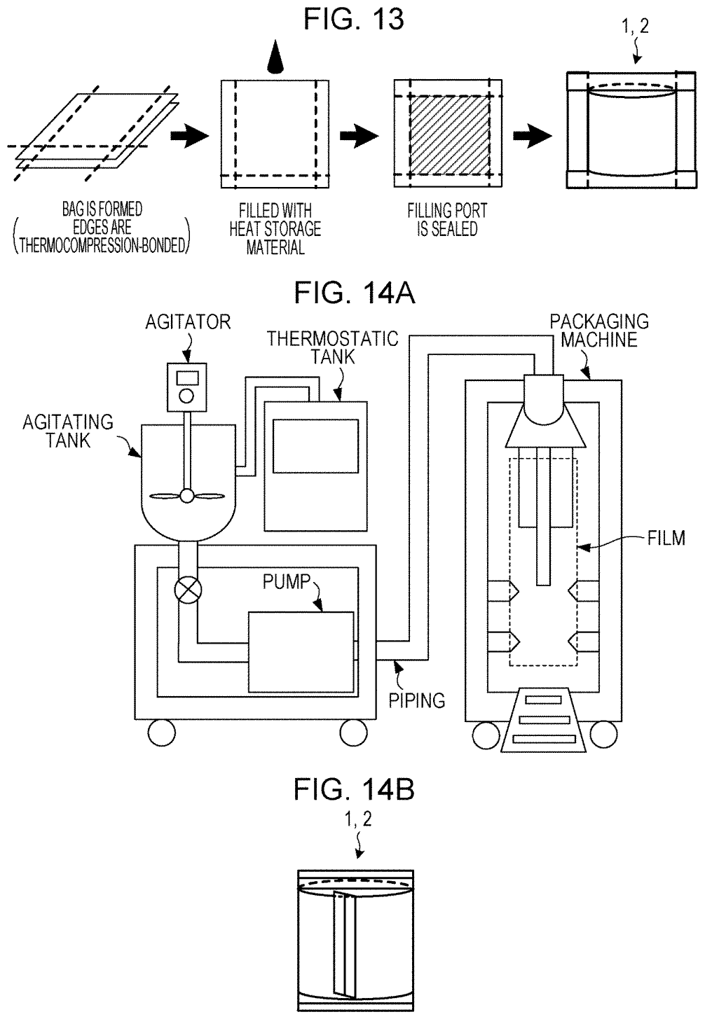

FIG. 13 is an illustration showing a production method using hand sealing. At least one film is prepared. Next, edges of the films except for a filling port for a cold storage material are thermocompression-bonded using a hand sealer to form a bag. Next, the film formed into the bag is filled with the cold storage material (the freezable material or the non-freezable material). After the bag is filled with the cold storage material, the filling port is thermocompression-bonded to thereby produce the cold storage layer or the buffer layer.

(b) Production Method Using Automatic Packaging Machine

FIGS. 14A and 14B are illustrations showing a production method using an automatic packaging machine. A cold storage material is produced in an agitating tank. Next, a pump is actuated, and the cold storage material produced is packaged with a film using a vertical pillow-type packaging machine to produce the cold storage layer or the buffer layer. Specifically, while fixed into a cylindrical shape, the film is sealed by thermocompression bonding using the automatic packaging machine shown in FIG. 14A, and the cold storage layer or the buffer layer can thereby be produced. As shown in FIG. 14B, the number of sealed portions in the cold storage layer and the buffer layer produced is smaller than the number of sealed portions in those produced by hand sealing. Liquid leakage is likely to occur in a sealed portion. Therefore, the use of the automatic packaging machine that can produce the cold storage layer and the buffer layer with a smaller number of sealed portions allows prevention of leakage of the cold storage materials.

(2) Next, condition indicators each including a warning member or a temperature display member impregnated with an indicator are applied to the cold storage layer and the buffer layer produced in (a) or (b) using an adhesive, and the cooling tool is thereby produced.

The two examples (a) and (b) of the method for producing the cold storage layer and the buffer layer have been described. However, the production method and the production machines are not limited to those described above. Examples of the material of the packaging material for the cold storage layer and the buffer layer include, but not limited to, nylon, aluminum-evaporated nylon, and aluminum.

### (Placement Positions of Condition Indicators)

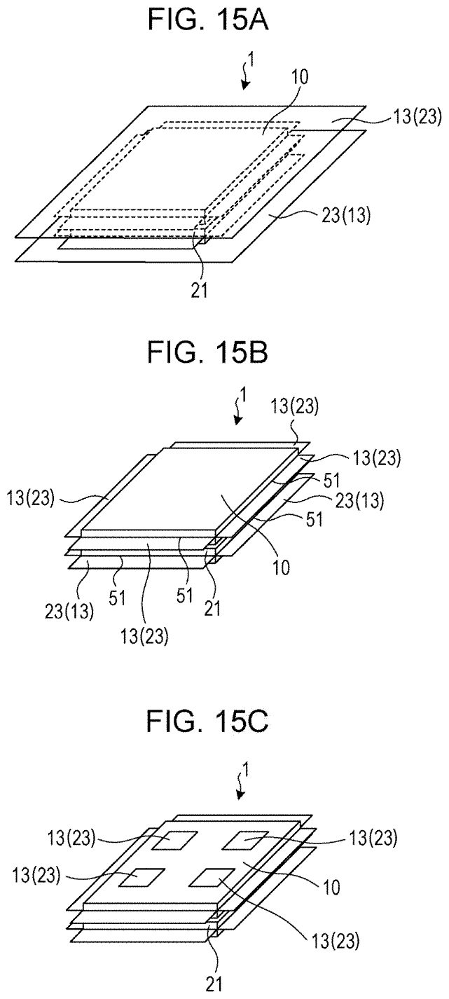

Next, the placement positions of the condition indicators will be described. Specifically, the placement positions of the warning members (the first warning member 13 and the second warning member 23) will be described as examples. FIGS. 15A to 15C are illustrations showing examples of the cooling tool 1 in which the warning members (the first warning member 13 and the second warning member 23) are disposed on the cold storage layer 10 and the buffer layer 21.

(a) FIG. 15A is an illustration showing an example of the cooling tool 1 in which the warning members (the first warning member 13 and the second warning member 23) are disposed on the upper and lower sides, with respect to a direction orthogonal to the drawing sheet, of the cold storage layer 10 and the buffer layer 21, respectively. By disposing the warning members (the first warning member 13 and the second warning member 23) so as to cover the cold storage layer 10 and the buffer layer 21 as described above, the warning members (the first warning member 13 and the second warning member 23) can change color immediately no matter where leakage of the freezable material or the non-freezable material occurs, and therefore the user can immediately notice the leakage of the freezable material or the non-freezable material.

(b) FIG. 15B is an illustration showing an example of the cooling tool 1 in which the warning members (first warning members 13 and second warning members 23) are disposed so as to surround the circumferences of the cold storage layer 10 and the buffer layer 21. Liquid leakage is like to occur in sealed portions 51, so that the warning members may be disposed mainly on the sealed portions 51.

(c) FIG. 15C is an illustration showing an example of the cooling tool 1 in which a plurality of warning members (first warning members 13 and second warning members 23) are disposed at different positions on the cold storage layer 10 and the buffer layer 21. As described above, a plurality of warning members (first warning members 13 and second warning members 23) of any size may be disposed at different positions on the cold storage layer 10 and the buffer layer 21.

Each warning member may be disposed at any position so long as leakage of the cold storage material or the non-freezable material can be visually checked. The placement positions of the warning members are not limited to those described above.

Each temperature display member may be placed at any position on the cold storage layer or the buffer layer so long as the temperature of the cold storage material or the non-freezable material can be measured and displayed to the user. However, it is preferable that the temperature display members are disposed in a manner similar to that for the warning members in FIG. 15A or 15C.

### (Protection of Condition Indicators)

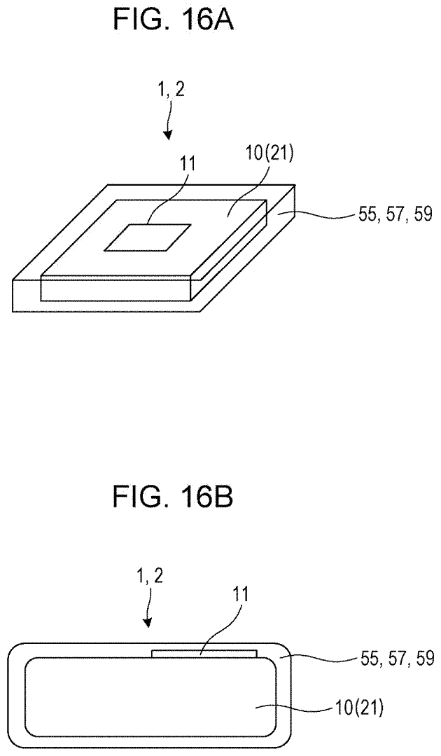

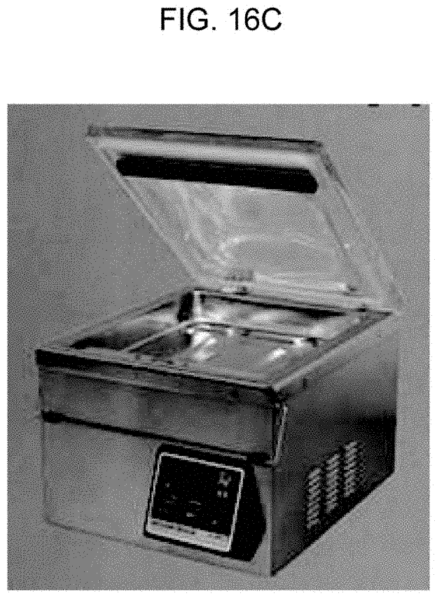

The condition indicators 11 are prone to deterioration and damage such as abrasion. Therefore, the cooling tool 1 may further include a film for protecting each condition indicator 11. A method for protecting the condition indicator 11 will be described. FIG. 16A is a perspective view schematically showing the cooling tool 1, 2 packed with a film, and FIG. 16B is a side view schematically showing the cooling tool 1, 2 packed with the film. The cooling tool 1, 2 may have a structure in which each of the cold storage layer 10 with a condition indicator 11 disposed thereon and the buffer layer 21 with a condition indicator 11 disposed thereon is packed with a film. Protection methods will next be described, but these are not limitations.

(1) Protection Method Using Vacuum Packing

A protection method using vacuum packing will be described. A vacuum-packed product can be produced using a small desktop vacuum packaging machine shown in FIG. 16C. (a) First, the cold storage layer 10 or the buffer layer 21 with the condition indicator 11 placed thereon is covered with a vacuum-packing film 55. (b) Next, the resulting product is sealed while evacuated. By sealing the product while evacuated, the gap between the vacuum-packing film 55 and the cold storage layer 10 or the buffer layer 21 can be as much as possible, and inhibition of heat transfer due to an air layer can be eliminated. In the structure described above, the condition indicator 11 is not exposed to the outside, and the vacuum-packing film 55 serving as the outermost layer is added. Therefore, the condition indicator 11 is prevented from deterioration and damage, and the strength of the cold storage layer 10 or the buffer layer 21 is improved.

(2) Protection Method Using Heat Shrinking Packing

A protection method using heat shrinking packing will be described. (a) First, the cold storage layer 10 or the buffer layer 21 with the condition indicator 11 placed thereon is covered with a heat shrinking packing film 55. (b) Next, heat is applied to the heat shrinking packing film 57 to shrink the heat shrinking packing film 57. (c) The heat shrinking packing film 57 is thereby caused to adhere to the cold storage layer 10 or the buffer layer 21 with the condition indicator 11 placed thereon to pack them. As described above, the heat shrinking packing film 57 shrinks so as to conform to the shape of the cold storage layer 10 or the buffer layer 21. Therefore, the gap between the heat shrinking packing film 57 and the cold storage layer 10 or the buffer layer 21 can be as much as possible, and inhibition of heat transfer due to an air layer can be eliminated. In the structure described above, the condition indicator 11 is not exposed to the outside, and the heat shrinking packing film 57 serving as the outermost layer is added. Therefore, the condition indicator 11 is prevented from deterioration and damage, and the strength of the cold storage layer 10 or the buffer layer 21 is improved.

(3) Protection Method Using Shrink Film

FIG. 16D is an illustration schematically showing the procedure of a protection method using a shrink film. (a) First, the cold storage layer 10 or the buffer layer 21 with the condition indicator 11 placed thereon is covered with a shrink film 59. To cover the cold storage layer 10 or the buffer layer 21 with the shrink film 59, a shrink label method in which the cold storage layer 10 or the buffer layer 21 is covered with a film shown in FIG. 16D or an ROSO method in which a film is wound around the cold storage layer 10 and the buffer layer 21 may be used. (b) Next, the shrink film 59 is heat-shrunk for packing. The shrink film 59 shrinks so as to conform to the shape of the cold storage layer 10 and the buffer layer 21, as in (2). Therefore, the gap between the shrink film 59 and the cold storage layer 10 or the buffer layer 21 can be as much as possible, and inhibition of heat transfer due to an air layer can be prevented. In the structure described above, the condition indicator 11 is not exposed to the outside, and the shrink film 59 serving as the outermost layer is added. Therefore, the condition indicator 11 is prevented from deterioration and damage, and the strength of the cold storage layer 10 or the buffer layer 21 is improved.

### Third Embodiment

### (Structure of Cooling Tool)

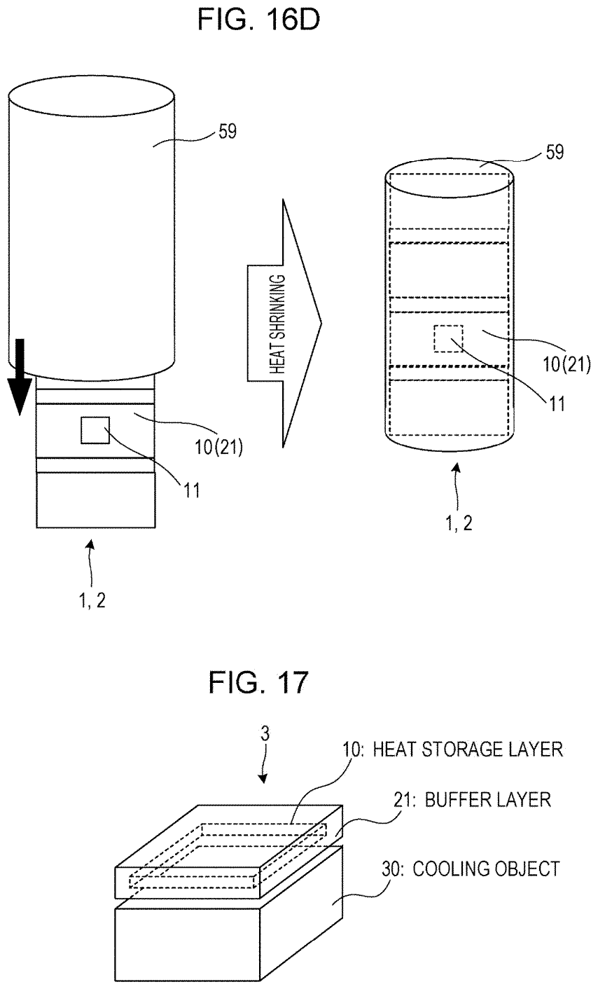

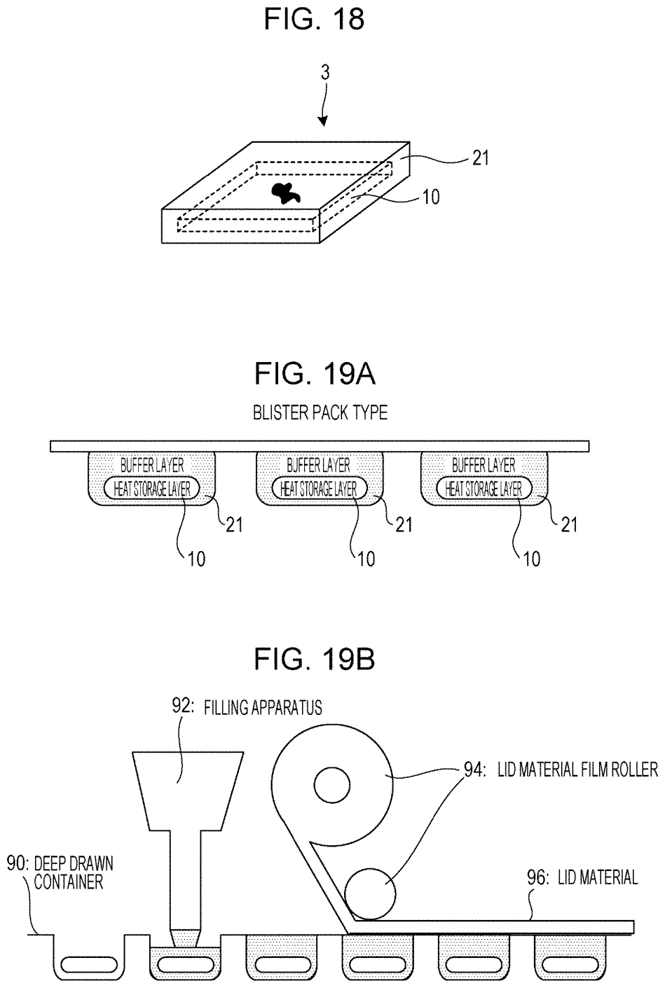

FIG. 17 is an illustration schematically showing a cooling tool 3 in a third embodiment. The cooling tool 3 in the third embodiment has a structure in which the cold storage layer 10 is contained within the buffer layer 21. An indicator is mixed into the non-freezable material forming the buffer layer 21. When the freezable material forming the cold storage layer 10 is broken by a stimulus and leaks from the cold storage layer 10 into the buffer layer 21 as shown in FIG. 18, the freezable material reacts with the indicator in the buffer layer 21, and the buffer layer 21 is colored, so that the user can be informed of the leakage of the freezable material. The freezable material, the non-freezable material, and the indicator used in the cooling tool 3 in the third embodiment are the same as those described in the first embodiment, and the description thereof will be omitted.

### (Method for Producing Cooling Tool)

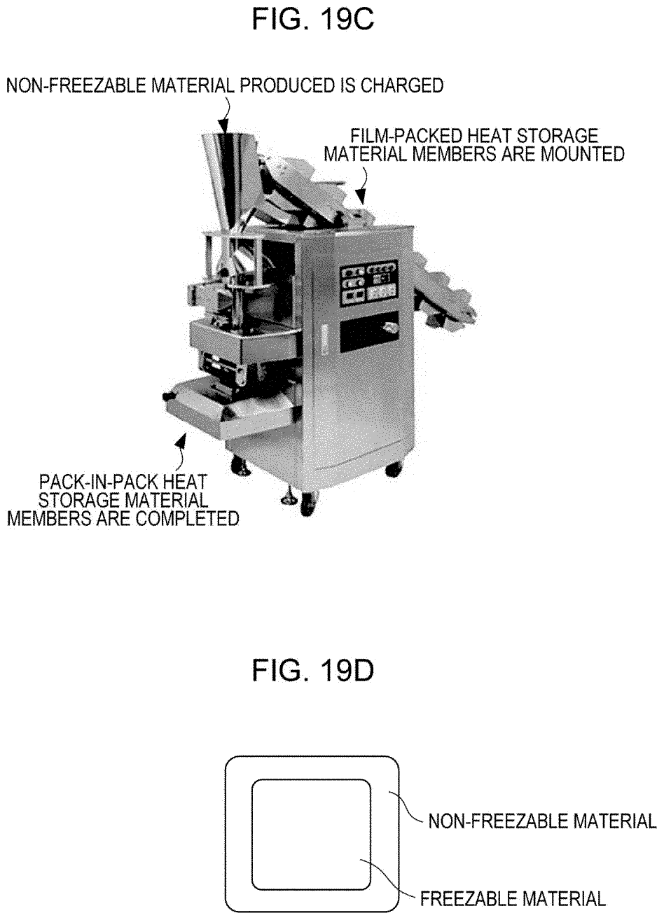

FIGS. 19A to 19D are illustrations showing a process of manufacturing the cooling tool according to the third embodiment. The cooling tool according to the third embodiment can be formed as a blister pack-type cooling tool. As shown in FIG. 19A, the cooling tool is packed such that the cold storage layer 10 is contained within the buffer layer 21. As shown in FIG. 19B, the cold storage layer 10 is placed in a deep drawn container 90, and a filling apparatus 92 is used to fill the non-freezable material into the deep drawn container 90. Then lid material film rollers 94 are used to supply a lid material 96 to thereby form a blister pack. Alternatively, a vertical pillow-type packaging machine shown in FIG. 19C may be used to produce a pack-in-pack shown in FIG. 19D and including the freezable material packed with a film and the non-freezable material that are packed with a film. The cooling tool may be produced using the production method using hand sealing described in the first embodiment.

### Fourth Embodiment

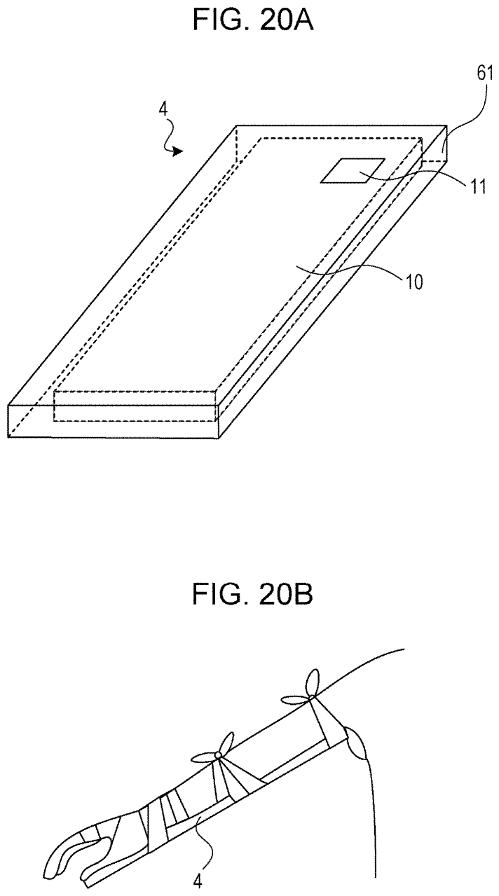

FIG. 20A is an illustration schematically showing a cooling tool 4 according to a fourth embodiment. In the cooling tool 4 according to the fourth embodiment, a plastic container 61 is filled with a cold storage material (freezable material), and a condition indicator 11 is disposed at a given position on the plastic container 61. FIG. 20B is an illustration showing an example of the use of the cooling tool 4 according to the fourth embodiment. When the cooling tool 4 is used, for example, for an arm fracture, the cooling tool 4 may be fitted to the arm so as to extend from the armpit toward the hand.

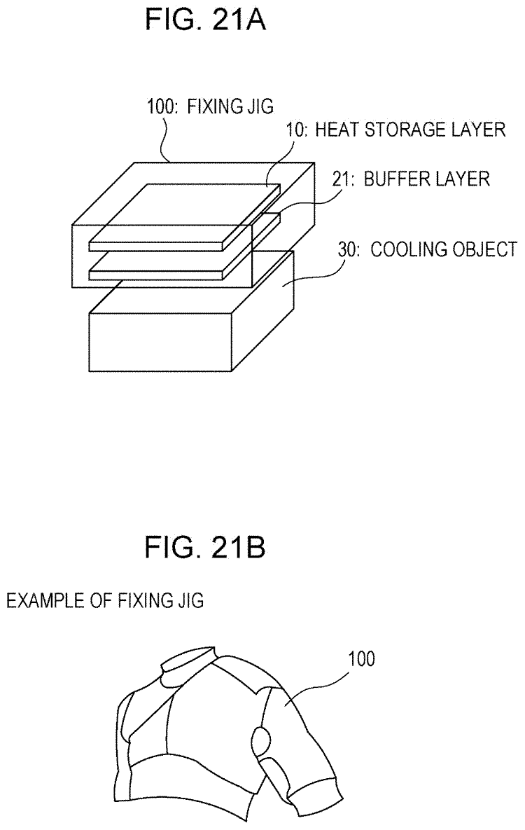

As shown in FIG. 21A, the cooling tool may be sealed in a fixing jig 100 used to fix the cooling tool to a cooling object. Examples of the fixing jig 100 include supporters and towels. FIG. 21B is an illustration showing an example of the use of the cooling tool. A leakage warning member may be mounted on the fixing jig 100.

One aspect of the present invention may have the following structure. Specifically, (A) a cooling tool according to the above aspect of the present invention is a cooling tool that keeps a cooling object cold by utilizing the latent heat of a freezable material. The cooling tool includes: a freezable material that undergoes phase change at a specific temperature; a cold storage layer including a first container portion that contains the freezable material; and a condition indicator that shows the physical condition of the freezable material.

As described above, by providing the condition indicator showing the physical condition of the freezable material, the user can judge at a glance the condition of the freezable material, e.g., whether the freezable material is leaking from the cold storage layer or whether the temperature of the freezable material is appropriate for keeping the cooling object cold, so that the cooling tool in an appropriate condition can be used.

(B) In the cooling tool according to the above aspect of the invention, the freezable material has an acidic, neutral, or alkaline character, and the condition indicator includes a first warning member that is located at least at a given position on the cold storage layer and impregnated with a first indicator whose color changes according to the pH of the freezable material.

When the freezable material comes into contact with the first warning member impregnated with the first indicator, the color of the first warning member is changed, so that the user can be immediately informed of the leakage of the freezable material from the cold storage layer.

(C) The cooling tool according to the above aspect of the invention further includes a buffer layer including: a non-freezable material that forms a liquid phase at a phase change temperature of the freezable material; and a second container portion that contains the non-freezable material.

Since the buffer layer including the non-freezable material that forms a liquid phases at the phase change temperature of the freezable material is disposed as described above, the adhesion between the cooling object and the cooling tool is improved, and this allows efficient heat transfer between the cooling object and the cold storage layer.

(D) In the cooling tool according to the above aspect of the invention, the non-freezable material has an acidic, neutral, or alkaline character, and the condition indicator includes a second warning member that is located at least at a given position on the buffer layer and impregnated with a second indicator whose color changes according to the pH of the non-freezable material.

When the non-freezable material comes into contact with the second warning member impregnated with the second indicator, the color of the second warning member is changed, so that the user can be immediately informed of the leakage of the non-freezable material from the buffer layer.

(E) In the cooling tool according to the above aspect of the invention, the cold storage layer is contained within the buffer layer. The buffer layer contains a first indicator whose color changes according to the pH of the freezable material, and the first indicator changes color in the case where the first indicator reacts with the freezable material leaking into the buffer layer.

When the cooling tool has a pack-in-pack structure in which the entire cold storage layer is contained within the buffer layer and the non-freezable material contains the indicator, the reaction of the indicator contained in the non-freezable material and the freezable material causes the indicator to change color within the buffer layer. Therefore, even when the cold storage layer that is likely to deteriorate due to repeated freezing and thawing of the freezable material breaks and the freezable material leaks into the buffer layer, the user can be informed of the leakage of the freezable material.

(F) In the cooling tool according to the above aspect of the invention, the freezable material has an acidic, neutral, or alkaline character, and the non-freezable material has an acidic, neutral, or alkaline character that differs from the character of the freezable material. The first indicator changes color at the pH of the freezable material.

When the buffer layer contains a solution mixture composed of the indicator and the non-freezable material and having the same pH as the freezable material, the color of the solution mixture can be changed. In this case, even when the cold storage layer that is likely to deteriorate due to repeated freezing and thawing of the freezable material breaks and the freezable material leaks into the buffer layer, the user can be informed of the leakage of the freezable material.

(G) In the cooling tool according to the above aspect of the invention, the condition indicator includes a temperature display member disposed at least at a given position on the cold storage layer and displays the temperature of the freezable material.

When the temperature display member is disposed at the given position on the cold storage layer, the temperature state of the cold storage layer can be known at a glance, and the cooling tool can be used at suitable temperature. Therefore, damage to the cooling object that occurs when the temperature of the cold storage layer is excessively low can be prevented, and a reduction in the cooling performance of the cooling object that occurs when the temperature of the cold storage layer is equal to or higher than the temperature of the cooling object can be prevented.

(H) In the cooling tool according to the above aspect of the invention, the condition indicator includes a temperature display member that is disposed at least at a given position on the buffer layer and displays the temperature of the non-freezable material.

When the temperature measuring member is disposed at the given position on the buffer layer, the temperature state of the buffer layer can be known at a glance, and the cooling tool can be used at suitable temperature. Therefore, damage to the cooling object that occurs when the temperature of the buffer layer is excessively low can be prevented, and a reduction in the cooling performance of the cooling object that occurs when the temperature of the cold storage layer is equal to or higher than the temperature of the cooling object can be prevented.

(I) in the cooling tool according to the above aspect of the invention, the temperature display member is formed of a thermochromic material whose color changes with temperature.

Since the temperature state can be represented by color, the temperature state can be easily known, and whether the cooling tool has a suitable temperature and is usable can be immediately judged before the cooling tool is used.

As has been described above, in the above aspect of the present invention, the user can judge at a glance the condition of the freezable material, e.g., whether the freezable material is leaking from the cold storage layer or whether the temperature of the freezable material is appropriate for keeping the cooling object cold, so that the cooling tool in an appropriate condition can be used.

The present international application claims priority from Japanese Patent Application No. 2016-227269 filed on Nov. 22, 2016, and the entire contents of Japanese Patent Application No. 2016-227269 are incorporated herein by reference.

### REFERENCE SIGNS LIST

1, 2, 3, 4 cooling tool

10 cold storage layer

11 condition indicator

13 first warning member, warning member

21 buffer layer

23 second warning member, warning member

25 temperature display member

25a low-temperature-side temperature display member

25b high-temperature-side temperature display member

27 label

30 cooling object

51 sealed portion

55 vacuum-packing film

57 heat shrinking packing film

59 shrink film

61 plastic container

90 deep drawn container

92 filling apparatus

94 lid material film roller

96 lid material

100 fixing jig

1. A cooling tool that keeps a cooling object cold by utilizing the latent heat of a freezable material, the cooling tool comprising:
a freezable material that undergoes phase change at a specific temperature;
a heat storage layer including a first container portion that contains the freezable material; and
a condition indicator that shows a physical condition of the freezable material.

2. The cooling tool according to claim 1,
wherein the freezable material has an acidic, neutral, or alkaline character, and
wherein the condition indicator includes a first warning member that is located at least at a given position on the heat storage layer and impregnated with a first indicator whose color changes according to the pH of the freezable material.

3. The cooling tool according to claim 1, further comprising a buffer layer including:
a non-freezable material that forms a liquid phase at a phase change temperature of the freezable material; and
a second container portion that contains the non-freezable material.

4. The cooling tool according to claim 3,
wherein the non-freezable material has an acidic, neutral, or alkaline character, and
wherein the condition indicator includes a second warning member that is located at least at a given position on the buffer layer and impregnated with a second indicator whose color changes according to the pH of the non-freezable material.

5. The cooling tool according to claim 3,
wherein the heat storage layer is contained within the buffer layer, and
wherein the buffer layer contains a first indicator whose color changes according to the pH of the freezable material, and the first indicator changes color in the case where the first indicator reacts with the freezable material leaking into the buffer layer.

6. The cooling tool according to claim 5,
wherein the freezable material has an acidic, neutral, or alkaline character,
wherein the non-freezable material has an acidic, neutral, or alkaline character that differs from the character of the freezable material, and
wherein the first indicator changes color at the pH of the freezable material.

7. The cooling tool according to claim 1,
wherein the condition indicator includes a temperature display member disposed at least at a given position on the heat storage layer and displays the temperature of the freezable material.

8. The cooling tool according to claim 3,
wherein the condition indicator includes a temperature display member that is disposed at least at a given position on the buffer layer and displays the temperature of the non-freezable material.

9. The cooling tool according to claim 7,
wherein the temperature display member is formed of a thermochromic material whose color changes with temperature.
